(19) United States
(12) Patent Application Publication
(10) Pub. No.: US 2017/0318837 A1
(43) Pub. Date: Nov. 9, 2017

(54) Method and Composition for Reducing Hyperthyroidism

(71) Applicant: Hill's Pet Nutrition, Inc., Topeka, KS (US)

(72) Inventors: Dennis JEWELL, Lawrence, KS (US); Xiangming GAO, Topeka, KS (US)

(73) Assignee: Hill's Pet Nutrition, Inc., Topeka, KS (US)

(21) Appl. No.: 15/533,248

(22) PCT Filed: Dec. 17, 2014
(86) PCT No.: PCT/US2014/070989

§ 371 Date: Jun. 5, 2017

(51) Int. Cl. A23K 50/40; A61K 9/00; A23K 40/25; A61K 31/122; A23K 20/174

(52) U.S. Cl. CPC A23K 50/40; A61K 31/122; A23K 20/174; A23K 40/25; A61K 9/0056

(57) ABSTRACT

The current invention relates to methods of reducing hyperthyroidism in an animal by feeding the animal with a diet including vitamin K. The vitamin K is in an amount effective to reduce factors such as creatinine, triiodothyronine (T3), and thyroid stimulating hormone (TSH). The current invention also relates to pet food composition that include effective amount of vitamin K to reduce the circulating levels of creatinine, T3 and TSH. In addition, the methods of making such a food composition are also disclosed.

### BACKGROUND

Hyperthyroidism is a common endocrine disorder in pets, especially older pets, such as cats over 10 years of age. Hyperthyroidism results from an overproduction of thyroid hormones and other factors involved in the expression of thyroid hormone, due to diseases, e.g. tumor, of the thyroid gland. The symptoms of hyperthyroidism are varied but may include increased appetite (but in some cases decreased), weight loss (fat and muscle) in spite of a good appetite, increased activity, increased drinking and urination, vomiting, diarrhea, heart disease (e.g. increased heart rate), increased respiration rate, hair coat/skin/nail abnormalities, nighttime yowling/restlessness/confusion behavior, or high blood pressure (hypertension). Hyperthyroidism may have severe short and long term consequences to the pet's health.

The current treatment options available to pets with hyperthyroidism often involves chronic administration of anti-thyroid drugs, surgical removal of one or both of the thyroid glands, and use of radioactive iodine to destroy the glandular tissue. However, each of these interventions has limitations and side effects. Therefore, it is desirable to develop new approaches to manage and reduce hyperthyroidism. Furthermore, it is desirable to manage and reduce hyperthyroidism in pets with dietary supplements.

### BRIEF SUMMARY

The current invention relates to a method of reducing hyperthyroidism in an animal in need thereof, comprising feeding the animal a diet comprising vitamin K in an amount effective to reduce circulating levels of one or more factors selected from the group consisting of creatinine, triiodothyronine (T3), and thyroid stimulating hormone (TSH).

The current invention also relates to a food composition comprising vitamin K in an amount effective to reduce circulating levels of one or more factors in an animal when the animal consumes the food composition, wherein the one or more factors are selected from the group consisting of creatinine, triiodothyronine (T3), and thyroid stimulating hormone (TSH).

The current invention also relates to a method for making a pet food composition comprising (a) preconditioning by mixing wet and dry ingredients at elevated temperature to form a kibble dough; (b) extruding the kibble dough at a high temperature and pressure; (c) drying the extruded kibble; and (d) enrobing the dried kibble with topical liquid and/or dry ingredients; wherein vitamin K is applied to the kibble at step (a) and/or (d), in an amount effective to reduce circulating levels of one of more factors in an animal when the animal consumes the food composition, and the one or more factors are selected from the group consisting of creatinine, triiodothyronine (T3), and thyroid stimulating hormone (TSH).

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of certain embodiment(s) is merely exemplary in nature and is in no way intended to limit the invention, its application, or uses. As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

As used herein, the term “animal” means any non-human organism belonging to the kingdom Animalia. The term “pet” means a domestic animal including but not limited to domestic dogs, cats, horses, cows, ferrets, rabbits, pigs, rats, mice, gerbils, hamsters, horses, minks, and the like. Domestic dogs and cats are particular examples of pets. It will be appreciated by one of skill in the art that some pets have different nutritional needs and some pets have similar nutritional needs.

As used herein, the term “hyperthyroidism” refers to a condition in which the thyroid gland produces and secretes excessive amounts thyroid hormones such as but not limited triiodothyronine (T3) and/or thyroxine (T4) in an animal. Hyperthyroidism in animals such as cats can be diagnosed and measured as to severity according methods and disease characteristics known in the art. (See, for example, Peterson et al., in The cat: diseases and clinical management, R. G. Sherding, Ed., New York, Churchill Livingstone, 2nd Edition, pp. 1416-1452, 1994; Gerber et al. Vet Clin North Am Small Anim Pract 24:541-65, 1994).

The term “T3” refers to the iodine-containing amino acid 3,5,3′-triiodothyronine. The term “free T3” refers to T3 that is not bound to a carrier protein such as thyroid-binding globulin, albumin, prealbumin, and the like.

As used herein, the term “TSH” refers to thyroid stimulating hormone, a glycoprotein hormone synthesized and secreted by thyrotrope cells in the anterior pituitary gland, which regulates the endocrine function of the thyroid gland, which produces T3 and T4 (thyroxine).

As used herein, the term “creatinine” refers to 2-Amino-1-methyl-1H-imidazol-4-ol, a breakdown product of creatine phosphate in muscle.

As used herein, the term “circulating” refers to the status of certain factors when the factors are moving with the blood and/or other body fluids, such as but not limited to circulating within the lymphatic system, of an animal.

As used herein, the term “vitamin K” refers to one or more of a group of structurally similar, fat-soluble vitamins known as the vitamin K family. The vitamin K family includes but is not limited to 2-methyl-1,4-naphthoquinone (3-) derivatives such as but not limited to vitamin K1 and vitamin K2.

As used herein, unless otherwise stated for a particular parameter, the term “about” refers to a range that encompasses an industry-acceptable range for inherent variability in analyses or process controls, including sampling error. Consistent with the Model Guidance of AAFCO, inherent variability is not meant to encompass variation associated with sloppy work or deficient procedures, but, rather, to address the inherent variation associated even with good practices and techniques.

As used here, the term “diet” refers to a regulated selection of food and drink for an animal. A diet may comprise a fixed or varied combination or food and/or drink compositions. The diet of the present invention may comprise the food composition of the present invention. The food composition of the present invention may comprise the ingredients and component of the diet herein disclosed.

Food compositions can be provided to an animal, such as but not limited to a pet, in the form of pet food. A variety of commonly known types of pet foods are available to pet owners. The selection of pet food includes but is not limited to wet pet food, semi-moist pet food, dry pet food and pet treats. Wet pet food generally has a moisture content greater than about 65%. Semi-moist pet food typically has a moisture content between about 20% and about 65% and may include humectants, potassium sorbate, and other ingredients to prevent microbial growth (bacteria and mold). Dry pet food such as but not limited to trod kibbles generally has a moisture content below about 15%. Pet treats typically may be semi-moist, chewable treats; dry treats in any number of forms; chewable bones or baked, extruded or stamped treats; confection treats; or other kinds of treats as is known to one skilled in the art.

As used herein, the term “kibble” or “food kibble” refers to a particulate pellet like component of animal feeds, such as dog and cat feeds. In some embodiments, a food kibble has a moisture, or water, content of less than 15% by weight. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

As used herein, the term “extrude” or “extrusion” refers to the process of sending preconditioned and/or prepared ingredient mixtures through an extruder. In some embodiments of extrusion, food kibbles are formed by an extrusion processes wherein a kibble dough, including a mixture of wet and dry ingredients, can be extruded under heat and pressure to form the food kibble. Any type of extruder can be used, examples of which include but are not limited to single screw extruders and twin-screw extruders. The list of sources, ingredients, and components as described hereinafter are listed such that combinations and mixtures thereof are also contemplated and within the scope herein.

The current invention relates to a food composition comprising vitamin K in an amount effective to reduce circulating levels of one or more factors in an animal when the animal consumes the food composition, wherein the one or more factors are selected from the group consisting of creatinine, triiodothyronine (T3), and thyroid stimulating hormone (TSH).

In addition, the current invention also relates to a method of reducing hyperthyroidism in an animal in need thereof, comprising feeding the animal a diet comprising vitamin K in an amount effective to reduce circulating levels of one or more factors selected from the group consisting of creatinine, triiodothyronine (T3), and thyroid stimulating hormone (TSH).

In some embodiments, the animal is a pet. In specific embodiments, the animal is a cat, such as but not limited to a domesticated house cat. In more specific embodiments, the animal is a cat older than 1, 2, 3, 4, 5, 6, 7, 8, 9, 10, 11, 12, 13, 14, or 15 years.

As used herein, the term “reduction” or “reduce” in the context of hyperthyroidism is used to refer, for example, to a decrease of circulating levels of one or more factors in an animal over time during which the animal consumes the food composition containing effective amount of vitamin K of the present invention compared to the circulating levels of the one or more factors in the same animal before the consumption of the food composition containing the effective amount of vitamin K.

The method may further comprise measuring the levels of the one or more factors in the animal prior to feeding the animal the diet comprising vitamin K. In some embodiments, baseline levels of the one or more factors in the animal are established. In one embodiment, the baseline levels are measurements of the circulating levels of the one or more factors. In one embodiment, the baseline levels are averages of a number of measurements for the circulating levels of each of the one or more factors.

The method may further comprise measuring the circulating levels of the one or more factors in the same animal after the animal consumes the diet comprising vitamin K at different time points. Moreover, the method may further comprise comparing the baseline circulating levels of the one or more factors in the animal prior to feeding the animal the diet comprising effective amount of vitamin K to the circulating levels of the one or more factors in the same animal after the animal consumes the diet comprising effective amount of vitamin K for a period of time. According to the present invention, the vitamin K in the diet is effective to reduce the circulating levels of the one or more factors, such as but not limited to creatinine, T3 and TSH.

In some embodiments of the inventions, the amount of the vitamin K in the diet is effective to reduce circulating levels of creatinine, but not free T3 or TSH. In some embodiments, the amount of the vitamin K in the diet is effective to reduce circulating levels of creatinine and free T3, but not TSH. In some embodiments, the amount of the vitamin K in the diet is effective to reduce circulating levels of creatinine and TSH, but not free T3. In some embodiments, the amount of the vitamin K in the diet is effective to reduce circulating levels of creatinine, free T3 and TSH. In some embodiments, the amount of the vitamin K in the diet is effective to reduce circulating levels of free T3, but not creatinine or TSH. In some embodiments, the amount of the vitamin K in the diet is effective to reduce circulating levels of free T3 and TSH, but not creatinine. In some embodiments, the amount of the vitamin K in the diet is effective to reduce circulating levels of TSH, but not creatinine or free T3.

The circulating levels of creatinine may be measured with methods known in the art, such as but not limited to an enzymatic colorimetric method, e.g. a method using the Roche Diagnostic's Cobas 6000 Analyzer Series. The circulating levels of free T3 may be measured with radioimmunoassay (RIA), such as but not limited to the method disclosed by Werner S. et al. J. Clin. Endocrinol. Metab. 38 (3): 493-5 (1974), which is incorporated herein by reference. The circulating levels of TSH may be measured with radioimmunoassay (RIA), such as but not limited to the method disclosed by Spencer C. et al. Clin Can Ada. 108 (3): 415-24 (1980), which is incorporated herein by reference.

In some embodiments, the methods of the present invention may comprise treating hyperthyroidism in an animal in need thereof, comprising feeding the animal a diet comprising vitamin K in an amount effective to reduce circulating levels of one or more factors selected from the group consisting of creatinine, triiodothyronine (T3), and thyroid stimulating hormone (TSH). In some embodiments, the method may further comprise identifying animals with hyperthyroidism prior to feeding the animals with the diet comprising vitamin K. In some embodiments, the animals with hyperthyroidism are identified by the baseline levels of factors such as but not limited to creatinine, free T3 and TSH.

In some embodiments, the methods of the present invention may comprise reducing the likelihood of developing hyperthyroidism in an animal, comprising feeding the animal a diet comprising vitamin K in an amount effective to reduce circulating levels of one or more factors selected from the group consisting of creatinine, triiodothyronine (T3), and thyroid stimulating hormone (TSH).

The vitamin K in the diet may be in an amount effective to reduce the circulating levels of creatinine, free T3 or TSH in an animal after the animal consumes the diet for a period of time. For example, the amount of vitamin K in the diet may be effective to reduce the circulating levels of creatinine, free T3 or TSH in an animal after the animal consumes the diet comprising effective amount of vitamin K for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 101, 105, 110, 113, 115, 120, 125, 130, 135, 140, 145, 150, 155, 160, 165, 170, 175, 180, 185, 190, 195 or 200 days. In some embodiments, the amount of vitamin K in the diet may be effective to reduce the circulating levels of creatinine, free T3 or TSH in an animal after the animal consumes the diet comprising effective amount of vitamin K for within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 101, 105, 110, 113, 115, 120, 125, 130, 135, 140, 145, 150, 155, 160, 165, 170, 175, 180, 185, 190, 195 or 200 days.

In some embodiments, the vitamin K in the diet may be about or more than about 0.01 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1 mg/kg, 1.5 mg/kg, 2 mg/kg, 2.5 mg/kg or 3 mg/kg of total food by weight.

In some embodiments, through the consumption of the vitamin K containing food, the animal may have a daily intake of vitamin K that effectively reduces circulating levels of creatinine, free T3 or TSH, and the daily intake of vitamin K may be in an amount of about or more than about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 microgram per day. In some embodiments, through the consumption of the vitamin K containing food, the animal may have a daily intake of vitamin K that effectively reduces circulating levels of creatinine, free T3 or TSH, and the daily intake of vitamin K may be in an amount of about 1-100, 1-90, 1-80, 1-70, 1-60, 1-50, 1-40, 1-30, 1-25, 1-20, 1-15, 1-10, 1-5, 5-100, 5-90, 5-80, 5-70, 5-60, 5-50, 5-40, 5-30, 5-25, 5-20 5-15, 5-10, 10-100, 10-90, 10-80, 10-70, 10-60, 10-50, 10-40, 10-30, 10-25, 10-20, or 10-15 microgram per day.

In some embodiments, the vitamin K in the diet is in an amount effective to reduce the circulating levels of creatinine in the animal. For example, after consuming the diet comprising effective amount of vitamin K for a period of time, the circulating levels of creatinine in the animal may be reduced by at least about 5%, 6%, 7%, 8%, 9%, 10%, 11%, 12%, 13%, 14%, 15%, 16%, 17%, 18%, 19%, 70%, 21%, 22%, 23%, 24%, 25%, 26%, 27%, 78%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, or 95% compared to circulating levels of creatinine prior to consumption of the diet comprising effective amount of vitamin K. In one embodiment, the amount of vitamin K in the diet is effective to reduce the circulating levels of creatinine by at least about 30%. In one specific embodiment, the amount of vitamin K in the diet is effective to reduce the circulating levels of creatinine by at least about 30% after the animal consumes the diet comprising the effective amount of vitamin K for at least a period of time selected from the group consisting of 25, 50, 75, 100 and 125 days. In a more specific embodiment, the amount of vitamin K in the diet is effective to reduce the circulating levels of creatinine by at least about 30% after the animal consumes the diet comprising the effective amount of vitamin K for at least about 100 days.

In some embodiments, the vitamin K in the diet is in an amount effective to reduce the circulating levels of free T3 in the animal. For example, after consuming the diet comprising effective amount of vitamin K for a period of time, the circulating levels of T3 in the animal may be reduced by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, or 95% compared to circulating levels of free T3 prior to consumption of the diet comprising effective amount of vitamin K. In one embodiment, the amount of vitamin K in the diet is effective to reduce the circulating levels of free T3 by at least about 30%. In one specific embodiment, the amount of vitamin K in the diet is effective to reduce the circulating levels of free T3 by at least about 30% after the animal consumes the diet comprising the effective amount of vitamin K for at least a period of time selected from the group consisting of 25, 50, 75, 100 and 125 days. In a more specific embodiment, the amount of vitamin K in the diet is effective to reduce the circulating levels of free T3 by at least about 30% after the animal consumes the diet comprising the effective amount of vitamin K for at least about 100 days.

In some embodiments, the vitamin K in the diet is in an amount effective to reduce the circulating levels of TSH in the animal. For example, after consuming the diet comprising effective amount of vitamin K for a period of time, the circulating levels of TSH in the animal may be reduced by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, or 95% compared to circulating levels of TSH prior to consumption of the diet comprising effective amount of vitamin K. In one embodiment, the amount of vitamin K in the diet is effective to reduce the circulating levels of TSH by at least about 80%. In one specific embodiment, the amount of vitamin K in the diet is effective to reduce the circulating levels of TSH by at least about 80% after the animal consumes the diet comprising the effective amount of vitamin K for at least a period of time selected from the group consisting of 25, 50, 75, 100 and 125 days. In a more specific embodiment, the amount of vitamin K in the diet is effective to reduce the circulating levels of TSH by at least about 80% after the animal consumes the diet comprising the effective amount of vitamin K for at least about 100 days.

In one embodiment, the amount of vitamin K in the diet is effective to reduce the circulating levels of creatinine by at least about 30%, the circulating levels of T3 by at least about 30%, and the circulating levels of TSH by at least about 80%. In one specific embodiment, the amount of vitamin K in the diet is effective to reduce the circulating levels of creatinine by at least about 30%, the circulating levels of T3 by at least about 30%, and the circulating levels of TSH by at least about 80% after the animal consumes the diet comprising the effective amount of vitamin K for at least about 100 days.

The food composition of the present invention may comprise vitamin K. In some embodiments, the vitamin K may be about or less than about 0.0001%, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 20%, or 25% of the total food composition by weight. In some embodiments, the vitamin K may be more than about 0.0001%, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 20%, or 25% of the total food composition by weight.

The food composition containing effective amount of vitamin K may be combined or mixed with food composition that does not contain vitamin K. For example, the food composition containing effective amount of vitamin K may be more than about 1%, 5%, 10%, 15%, 20%, 25%, 30%, 35%, 40%, 45%, 50%, 55%, 60%, 65%, 70%, 75%, 80%, 85%, 90%, 95% or 99% of the total food composition by weight. In some embodiments, the food composition containing effective amount of vitamin K may be less than about 1%, 5%, 10%, 15%, 20%, 25%, 30%, 35%, 40%, 45%, 50%, 55%, 60%, 65%, 70%, 75%, 80%, 85%, 90%, 95%, 99% or 100% of the total food composition by weight. In some embodiments, the diet of the present invention may comprise the food composition comprising effective amount of vitamin K and other food compositions that do not comprise vitamin K.

The food composition containing effective amount of vitamin K may comprise different kinds of trod products. For example, the food composition containing effective amount of vitamin K may comprise one or more types of dry food (e.g. kibbles), semi-moist food or wet food. The different kinds of food products may comprise different amount of vitamin K and some of the food products may not comprise vitamin K. For example, a food composition may comprise dry food comprising vitamin K and semi-moist food that does not comprise vitamin K and/or we food that does not comprise vitamin K. In one embodiment, the dry food containing vitamin K may be more than about 1%, 5%, 10%, 15%, 20%, 25%, 30%, 35%, 40%, 45%, 50%, 55%, 60%, 65%, 70%, 75%, 80%, 85%, 90%, 95% or 99% of the total food composition by weight. In another embodiment, the dry food containing vitamin K may be less than about 5%, 10%, 15%, 20%, 25%, 30%, 35%, 40%, 45%, 50%, 55%, 60%, 65%, 70%, 75%, 80%, 85%, 90%, 95%, 99% or 100% of the total food composition by weight. In some embodiments, the dry food containing vitamin K may be combined or mixed with semi-moist food or wet food that also contain vitamin K, in the same or a different amount. In some embodiments, the dry food containing vitamin K may be more than about 1%, 5%, 10%, 15%, 20%, 25%, 30%, 35%, 40%, 45%, 50%, 55%, 60%, 65%, 70%, 75%, 80%, 85%, 90%, 95% or 99% of the total food composition by weight. In some embodiments, the dry food containing vitamin K may be less than about 5%, 10%, 15%, 20%, 25%, 30%, 35%, 40%, 45%, 50%, 55%, 60%, 65%, 70%, 75%, 80%, 85%, 90%, 95%, 99% or 100% of the total food composition by weight.

The current invention also relates to methods of making a pet food composition, wherein the food composition comprises vitamin K in an amount effective to reduce circulating levels of one or more factors in an animal after the animal consumes the food composition, wherein the one or more factors are selected from the group consisting of creatinine, triiodothyronine (T3), and thyroid stimulating hormone (TSH). The food composition may comprise one or more of dry food, semi-moist food and wet food.

In some embodiments, current invention also relates to methods of making a dry food composition, comprising (a) preconditioning by mixing wet and dry ingredients at elevated temperature to form a kibble dough; (b) extruding the kibble dough at a high temperature and pressure; (c) drying the extruded kibble; and (d) enrobing the dried kibble with topical liquid and/or dry ingredients; wherein vitamin K is applied to the kibble at step (a) and/or (d), in an amount effective to reduce circulating levels of one of more factors in an animal when the animal consumes the food composition, and the one or more factors are selected from the group consisting of creatinine, triiodothyronine (T3), and thyroid stimulating hormone (TSH).

In some embodiments, the vitamin K is applied to the kibble dough in step (a) by mixing with other ingredient to form the kibble dough. In some embodiments, the vitamin K is applied to the food composition at step (d) by adding the vitamin K to the topical liquid and/or dry ingredients.

The kibble dough can be prepared in any suitable means from any suitable ingredients, such as, for example, a protein source, a carbohydrate source, a fat source, and any other ingredients suitable for animal or pet nutrition.

Similarly, the topical liquid and/or dry ingredients that are used to enrobing the food composition can be prepared in any suitable means from any suitable ingredients, such as, tier example, a protein source, a carbohydrate source, a fat source, and any other ingredients suitable for animal or pet nutrition.

In some embodiments, the food composition of the present invention comprise one or more ingredients such as but not limited to flax, corn, rim brewers, pea, chicken, soybean, tomato, cellulose, wheat, beet, lysine, potassium chloride, methionine, sodium chloride, carrot, dicalcium phosphate, vitamin premix, carnitine, lipoic acid alpha, mineral premix, calcium carbonate, taurine, glucosamine hydrochloride, chondroitin sulfate, grain blend, lactic acid, choline chloride, grain blend, palatant, fish oil, coconut oil, vitamin E oil, starch, poultry, fish, dairy, pork, beef, lamb, venison, and rabbit.

In some embodiments, the food composition of the present invention comprise one or more amino acid such as but not limited to arginine, histidine, isoleucine, leucine, methionine, phenylala nine, threonine, tryptophan, valine, taurine, camitine, alanine, aspartate, cystine, glutamate, glutamine, glycine, proline, serine, tyrosine, and hydroxyproline.

In some embodiments, the food composition of the present invention comprise one or more fatty acids such as but not limited to laurie acid, myristic acid, palmitic acid, palmitoleic acid, margaric acid, margaroleic acid, stearic acid, oleic acid, linoleic acid, g-linolenic acid, a-linolenic acid, stearidonic acid, arachidic acid, gadoleic acid, DHGLA, arachidonic acid, eicossatetra acid, EPA, behenic acid, erucic acid, docosatetra acid, and DPA.

In some embodiments, the thod composition of the present invention comprise one or more macro nutrients such as but not limited to moisture, protein, fat, crude fiber, ash, dietary fiber, soluble fiber, insoluble fiber, raffinose, and stachyose.

In some embodiments, the food composition of the present invention comprise one or more micro nutrients such as but not limited to beta-carotene, alpha-lipoic acid, glucosamine, chondroitin sulfate, lycopene, lutein, and quercetin.

In some embodiments, the food composition of the present invention comprise one or more minerals such as but not limited to calcium, phosphorus, potassium, sodium, chloride, iron, copper, copper, manganese, zinc, iodine, selenium, selenium, cobalt, sulfur, fluorine, chromium, boron, and oxalate.

In some embodiments, the food composition of the present invention comprise one or more vitamins such as but not limited to vitamin A, vitamin D, vitamin E, vitamin K, vitamin C, thiamine, riboflavin, niacin, pyridoxine, pantothenic acid, folic acid, vitamin B12, biotin, and choline

### EXAMPLES

### Example 1

In vivo studies were conducted in cats to show that vitamin K can reduce hyperthyroidism. Cats were fed with vitamin K containing food and the circulating levels of creatinine, free T3 and TSH were measured before and after the consumption of the vitamin K containing food.

Eight cats with naturally occurring hyperthyroidism were included in the study. The cats were assigned to one of the two diets (Control and Test) and consumed the diets throughout the testing period of 113 days. The diet of the Test group comprised vitamin K in the form of MSBC (menadione sodium bisulfite complex) in an amount of 0.3 mg/kg total food by weight. Creatinine, free T3, and TSH were measured at day 1 and day 113. All cats were allowed to eat ad libitum for a 20 hour period each day and had water available at all times. The average daily intake of total food was about 52.6 g/day.

The results for circulating creatinine are shown in Table 1.

TABLE 1

| | | | | Creatinine | | |
|---|---|---|---|---|---|---|
| | Creatinine | Creatinine | | Change | Creatinine | |
| | (mg/dL) | (mg/dL) | | (mg/dL) | Change (%) | DLSM |
| | Day 1 | Day 113 | DF | Day 1-113 | Day 1-113 | Pr |
| Control | 0.8688 | 0.8775 | 7 | +0.00875 | +1% | 0.95 |
| Test | 1.11 | 0.7414 | 7 | −0.3686 | −33.2% | 0.0242 |

In Table 1, Control refers to cats that were fed with control food—Ibod that does not contain vitamin K; Test refers to cats that were fed with food comprising vitamin K. DF: degree of freedom; DLSM: differences of least squares means; Pr: probability. Circulating creatinine was measured by an enzymatic calorimetric method (Roche Diagnostics, Cobas® 6000 Analyzer Series c501 Module).

While circulating levels of creatinine was not affected in the control group, there was a significant reduction in circulating creatinine levels in the Test group.

The results fbr circulating free T3 are shown in Table 2.

TABLE 2

| | | | | Free T3 | Free T3 | |
|---|---|---|---|---|---|---|
| | Free T3 | Free T3 | | Change | Change | |
| | (mg/dL) | (mg/dL) | | (mg/dL) | (%) | DLSM |
| | Day 1 | Day 113 | DF | Day 1-113 | Day 1-113 | Pr |
| Control | 2.5625 | 2.1750 | 7 | −0.3875 | −15.12% | 0.3265 |
| Test | 2.84 | 1.7429 | 7 | −1.0971 | −38.63% | 0.0168 |

In Table 2, Control refers to cats that were fed with control food—food that does not contain vitamin K; Test refers to cats that were fed with food comprising vitamin K. DF: degree of freedom; DLSM: differences of least squares means; Pr: probability. Circulating free T3 was measured with radioimmunoassay (RIA) at Michigan State University.

While there was no significant improvement of circulating free T3 in the control group, the Test group showed a significant reduction of circulating free T3.

The results for circulating TSH are shown in Table 3.

TABLE 3

| | | | | TSH | TSH | |
|---|---|---|---|---|---|---|
| | TSH | TSH | | Change | Change | |
| | (mg/dL) | (mg/dL) | | (mg/dL) | (%) | DLSM |
| | Day 1 | Day 113 | DF | Day 1-113 | Day 1-113 | Pr |
| Control | 10.125 | 4.5 | 7 | −5.625 | −55.55% | 0.0123 |
| Test | 10.8 | 1.0 | 7 | −9.8 | −90.7% | 0.0005 |

In Table 3, Control refers to cats that were fed with control food food that does not contain vitamin K; Test refers to cats that were fed with food comprising vitamin K. DF: degree of freedom; DLSM: differences of least squares means; Pr: probability. Circulating TSH was measured with radioimmunoassay (RI) at Michigan State University.

The Test group showed a more significant reduction of circulating TSH compared to the Control group.

1. A method of reducing hyperthyroidism in an animal in need thereof, comprising feeding the animal a diet comprising vitamin K in an amount effective to reduce circulating levels of one or more factors selected from the group consisting of creatinine, triiodothyronine (T3), and thyroid stimulating hormone (TSH).

2. The method of claim 1, wherein the animal is a cat.

3. The method of claim 1, wherein the diet comprises an amount of vitamin K effective to reduce circulating levels of creatinine in the animal.

4. The method of claim 3, wherein the amount of vitamin K in the diet is effective to reduce circulating levels of creatinine by at least 30%, as compared to the circulating levels of creatinine in the animal before the animal is fed the diet.

5. The method of claim 3, wherein the amount of vitamin K in the diet is effective to reduce circulating levels of creatinine by at least 30% when the animal is fed with the diet for at least a period of time selected from the group consisting of 25 days, 50 days, 100 days and 125 days, as compared to the circulating levels of creatinine in the animal before the animal is fed the diet.

6. The method of claim 1, wherein the diet comprises an amount of vitamin K. effective to reduce circulating levels of TSR in the animal.

7. The method of claim 6, wherein the amount of vitamin K in the diet is effective to reduce circulating TSH levels by at least 80%, as compared to the circulating levels of TSH in the animal before the animal is fed the diet.

8. The method of claim 6, wherein the amount of vitamin K in the diet is effective to reduce circulating TSH levels by at least 80% when the animal is fed with the diet for at least a period of time selected from the group consisting of 25 days, 50 days, 100 days and 125 days, as compared to the circulating levels of TSH in the animal before the animal is fed the diet.

9. The method of claim 1, wherein the diet comprises an amount of vitamin K effective to reduce circulating levels of free T3 in the animal.

10. The method of claim 9, wherein the amount of vitamin K in the diet is effective to reduce circulating levels of free T3 by at least 30%, as compared to the circulating levels of free T3 in the animal before the animal is fed the diet.

11. The method of claim 9, wherein the amount of vitamin K in the diet is effective to reduce circulating levels of free T3 by at least 30% when the animal is fed with the diet for at least a period of time selected from the group consisting of 25 days, 50 days, 100 days and 125 days, as compared to the circulating levels of free T3 in the animal before the animal is fed the diet.

12-20. (canceled)
